(19) United States
(12) Patent Application Publication
(10) Pub. No.: US 2021/0255140 A1
(43) Pub. Date: Aug. 19, 2021

(54) FLUIDIC STACK AND REFERENCE ELECTRODE FOR SAMPLE COLLECTION AND ANALYSIS AND RELATED METHODS

(71) Applicant: FemtoDx, Beverly Hills, CA (US)

(72) Inventor: Pritiraj Mohanty, Los Angeles, CA (US)

(73) Assignee: FemtoDx, Beverly Hills, CA (US)

(21) Appl. No.: 17/159,778

(22) Filed: Jan. 27, 2021

Related U.S. Application Data
(60) Provisional application No. 62/966,483, filed on Jan. 27, 2020.

(51) Int. Cl. G01N 27/414; G01N 27/327; B01L 3/00

(52) U.S. Cl. CPC G01N 27/4145; C12Q 1/6825; B01L 3/5027; G01N 27/3278

(57) ABSTRACT

Some embodiments relate to a device with a fluidic stack that includes a filter, a fluidic block adjacent to the filter that includes a first channel through the fluidic block, a first layer adjacent to the fluidic block that includes at least a portion of a second channel that is in fluid communication with the first channel, and a sensor adjacent to the first layer and in fluid communication with the second channel. The device can include a fluid reservoir that includes one or more dimensions of the second channel and/or the sensor, and a reference electrode disposed within the fluid reservoir.

### RELATED APPLICATIONS

This application claims priority to U.S. Provisional Application No. 62/966,483, filed Jan. 27, 2020, which is incorporated herein by reference in its entirety.

### TECHNICAL FIELD

The techniques described herein relate generally to methods and apparatus for small-scale (e.g., nanoscale) sensors used to sense chemical or biological species, and in particular to devices and methods comprising a fluidic stack, reference electrode, or both.

### BACKGROUND

Chemical or biological sensors can include nanowires and/or other small-scale electrical devices that essentially serve as sensitive transducers that convert chemical activity of interest into corresponding electrical signals that can be used to accurately represent the chemical activity. The nanosensors can include one or more nanowires (e.g., which may have a tubular form). The nanowires can be fabricated such that once functionalized, their surface will interact with adjacent molecular entities, such as chemical species. The interaction of the nanowires with molecular entities can induce a change in a property (such as conductance) of the nanowire.

### SUMMARY

Some embodiments of the techniques described herein provide microfluidic components for biosensor devices. The microfluidic components can be arranged as a stack of components that allow a fluid to be introduced into the biosensor device, optionally processed (e.g., filtered), and provided to the sensor component inside of the biosensor device via the microfluidic components. The microfluidic components can define one or more dimensions of a fluidic channel, including multiple subsections of the fluidic channel, to guide the fluid from its introduction into the device to the sensor.

Some embodiments of the techniques described herein provide a reference electrode disposed within the fluidic channel of a biosensor device. The reference electrode can be arranged in the fluidic channel according to one or more configurations. For example, in some embodiments the reference electrode can be disposed on a top surface of the portion of the fluidic channel that contains the sensor. The top surface can be provided, at least partially, by a microfluidic component, such as the bottom side of a microfluidic block. In some embodiments, the side surfaces of the fluidic channel are provided, at least partially, by another layer of the microfluidics components, such as an adhesive layer. In some embodiments, the bottom of the fluidic channel is provided, at least partially, by a top surface of the sensor.

In one aspect, a device comprises: a filter; a fluidic block adjacent to the filter, the fluidic block comprising a top surface, a bottom surface, and a first channel passing through the fluidic block from the top surface and to the bottom surface; a layer adjacent to the fluidic block, the layer comprising at least a portion of a second channel, wherein the second channel is in fluid communication with the first channel; and a sensor adjacent to the layer and in fluid communication with the second channel is described.

In another aspect, a device comprises: a sensor comprising a top surface; a layer comprising a bottom surface; a fluid reservoir proximate the bottom surface of the layer and the top surface of the sensor; and a reference electrode disposed within the fluid reservoir is described.

In yet another aspect, a device comprises a fluidic stack and a fluidic chamber is described. In some embodiments the fluidic stack comprises a filter; a fluidic block adjacent to the filter, the fluidic block comprising a first surface, a second surface, and a first channel through the fluidic block from the first surface and to the second surface; a first layer adjacent to the fluidic block, the first layer comprising at least a portion of a second channel, wherein the second channel is in fluid communication with the first channel; and a sensor adjacent to the first layer and in fluid communication with the second channel, and in some embodiments, the fluidic chamber comprises the sensor, the sensor further comprising a third surface; a second layer comprising a fourth surface; a fluid reservoir proximate the third surface of the second layer and the fourth surface of the sensor; and a reference electrode disposed within the fluid reservoir.

In accordance with some embodiments, a method of analyzing a sample is described, the method comprising passing the sample through a filter to produce a serum; flowing the serum through a fluidic block adjacent to the filter, the fluidic block comprising a top surface, a bottom surface, and a first channel through which the serum flows; flowing the serum through a second channel of a layer, wherein the layer is adjacent to the fluidic block and wherein the second channel is in fluid communication with the first channel; and analyzing the serum in the second channel using a sensor and a reference electrode, wherein the second channel comprises at least a portion of the reference electrode and at least a portion of the sensor.

The subject matter described herein involves, in some cases, interrelated products, alternative solutions to a particular problem, and/or a plurality of different uses of one or more systems and/or articles.

Other advantages and novel features of the techniques will become apparent from the following detailed description of various non-limiting embodiments of the techniques when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the techniques described herein will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the techniques shown where illustration is not necessary to allow those of ordinary skill in the art to understand the techniques. In the figures:

FIG. 1A is a schematic diagram of an expanded view of fluidic stack and a sensor with a top housing and bottom housing before enclosure, according to some embodiments;

FIG. 1B is a schematic illustration of an expanded view of a microfluidic device comprising a fluidic stack and a sensor, according to some embodiments;

FIG. 1C is a schematic illustration of a microfluidic device enclosed by a top housing and a bottom housing, according to some embodiments;

FIG. 2A is a schematic illustration of a device comprising various components of a fluidic stack, according to some embodiments;

FIG. 2B is a schematic diagram of a first embodiment of an intervening channel of a fluidic block, according to one embodiment;

FIG. 2C is a schematic diagram of a second embodiment of an intervening channel of a fluidic block, according to one embodiment;

FIG. 3A is a schematic illustration of a first embodiment of a reference electrode disposed on a top surface of a fluid reservoir that includes a sensor, according to one set of embodiments;

FIG. 3B is a schematic illustration of a second embodiment of a reference electrode disposed on a bottom surface of a fluid reservoir that includes a sensor, according to one set of embodiments;

FIG. 3C is a schematic illustration of a third embodiment of a reference electrode disposed on an anchor connected to the bottom surface of a fluid reservoir, according to one set of embodiments;

FIG. 3D is a schematic illustration of a reference electrode disposed on a top surface of a fluid reservoir and further details of the sensor in the reservoir, according to one set of embodiments;

FIGS. 4A-4E schematically depict a fluidic stack and fluidic chamber configured to analyze a sample, according to some embodiments;

FIG. 5 is a schematic diagram illustrating the use of a sensor device used to detect species in an analyte solution, according to some examples;

FIG. 6 (consisting of parts 2(a)-2(d)) depicts a nanochannel-based sensing element in the circuit of FIG. 5, according to some examples;

FIG. 7 depicts a sensor employing an array of nanochannels, according to some examples; and

FIG. 8 is a schematic illustration of a reference electrode/anchor site proximate to a nanowire sensor/reference electrode array, according to one set of embodiments.

### DETAILED DESCRIPTION

Biosensor devices as described herein can include various components, including arrangements of microfluidic components (e.g., microfluidic stacks), sensors, and related electronic components for analyzing a sample (e.g., a fluid).

Devices and methods describe herein may be used for measuring the presence of an analyte (e.g., a protein, a hormone, an enzyme, etc.) within a sample. Non-limiting examples of samples include blood (e.g. a droplet of blood) or a serum. In some embodiments, the analyte is contained within the sample and devices and methods herein may allow for analysis of the sample or an analyte within the sample. In some embodiments, the device includes a series of microfluidic components that are used to control the flow of the sample, such as filters, a fluidic block, adhesive layers, and/or the like. For example, the fluidic block can include an intervening channel (e.g., a first channel, or a first portion of a fluidic channel) that passes through one side of the fluidic block to another side of the fluidic block. The microfluidic components can also include a layer (e.g., an adhesive layer) that provides one or more dimensions of another channel (e.g., a second channel, or a second portion of the fluidic channel) that is in fluid communication with the intervening channel and the sensor. In some embodiments, the sensor (e.g., a semiconductor) is configured to detect and/or analyze an analyte within a sample. For some embodiments, the sensor comprises a metal-oxide semiconductor field-effect transistor (MOSFET) configured to analyze a sample in an adjacent fluidic chamber, as is described further below and elsewhere herewithin. The fluidic stack and the sensor may function together in the analysis of a sample, for example, by isolating a serum from a sample of blood to allow the sensor to detect an analyte in the serum.

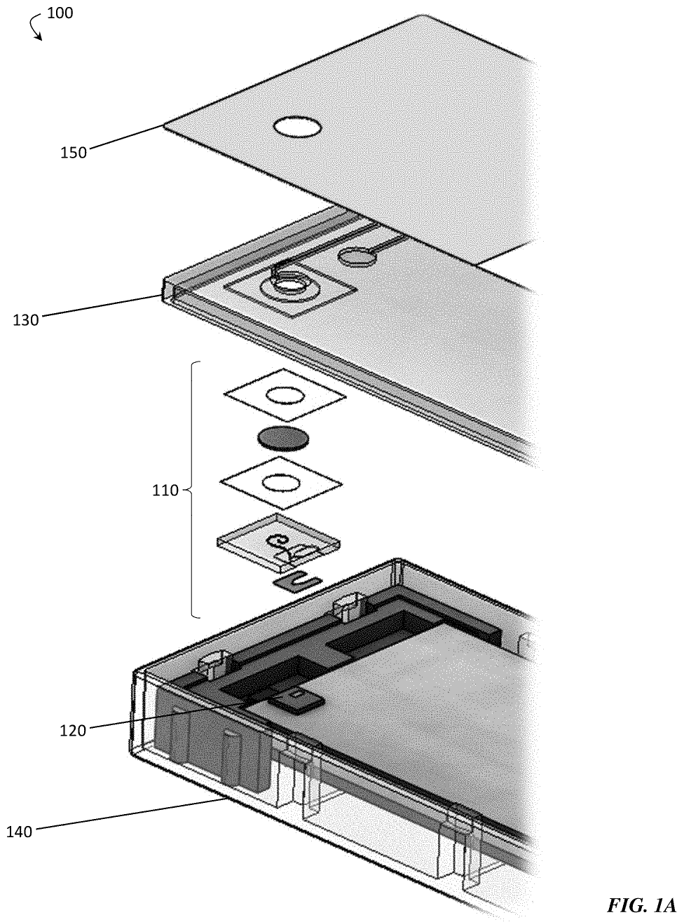

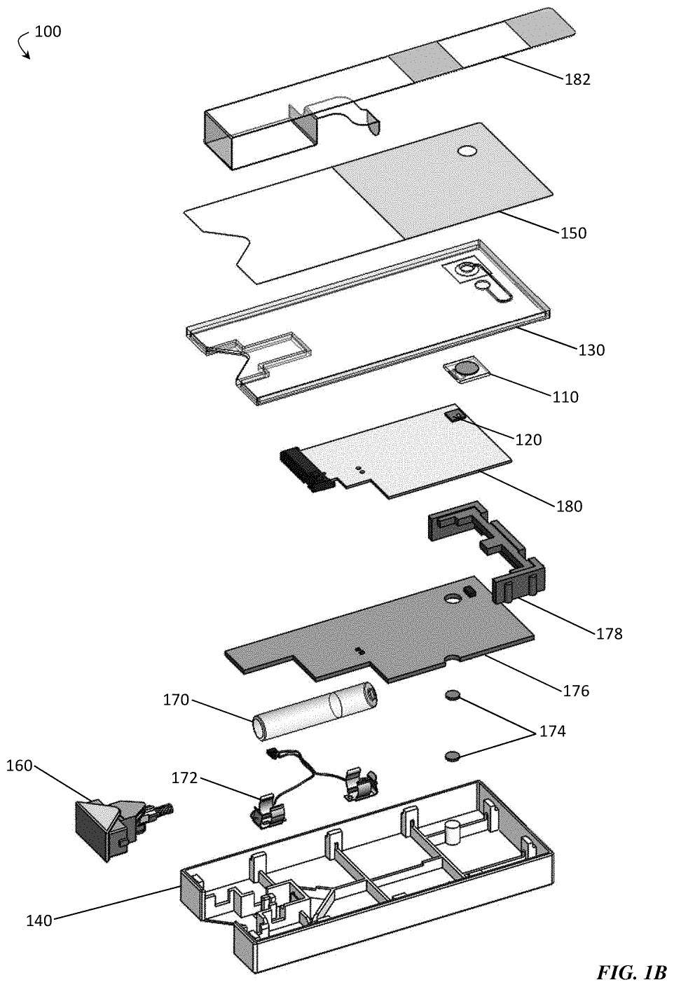

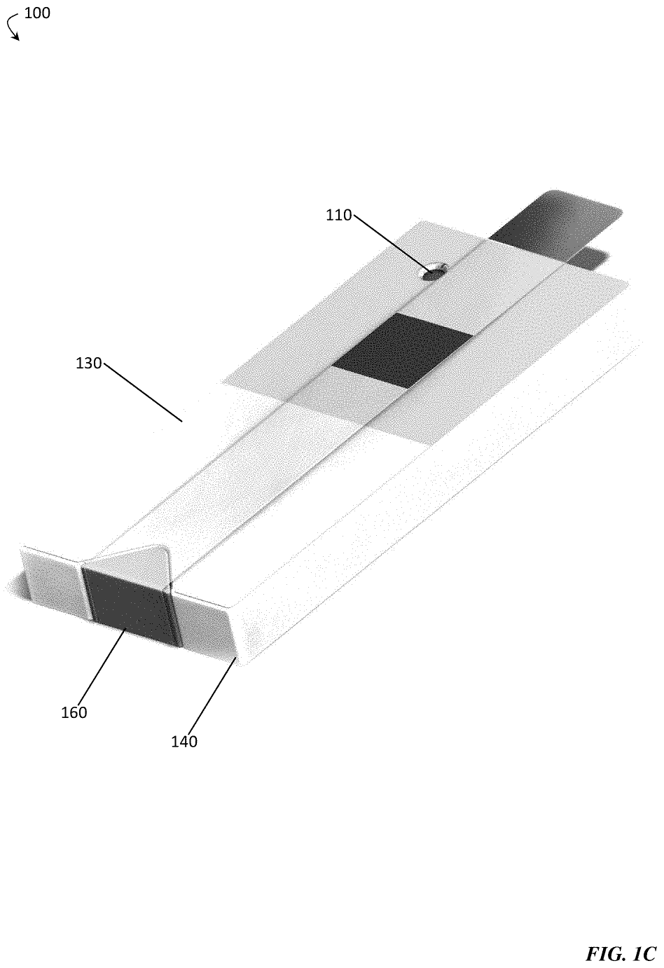

In accordance with some embodiments, a microfluidic unit (e.g., a microfluidic stack) may comprise various components as shown in FIG. 1A disposed within a sensor device. FIG. 1A shows sensing device 100, which comprises fluidic stack 110 and sensor 120. Fluidic stack 110 may comprise various components, such as a filter, a fluidic block, and/or additional layers, as described further below. In some embodiments, fluidic stack 110 is adjacent to sensor 120 as shown in FIG. 1A, which may allow a sample to pass through the fluidic stack to the sensor for analysis of the sample or a derivation of the sample. In some embodiments, fluidic stack 110 and sensor 120 may be enclosed by an enclosure. An expanded view of an exemplary enclosure is schematically illustrated in in FIG. 1A, where top housing 130 and bottom housing 140 of the enclosure may enclose fluidic stack 110 and sensor 120. The microfluidic device may also include laminate layer 150, adjacent to top housing 130.

In some embodiments, microfluidic device 100 may comprise additional features. Referring now to FIG. 1B, in some exemplary embodiments, microfluidic device 100 includes finger prick 160. In some embodiments, the finger prick is detachable and may contain a needle which may be used to prick a subject's (e.g., a patient's) finger in order to provide a sample of blood for analysis. In some embodiments, the finger prick 160 is optional and may not be present. Microfluidic device 100 may also contain battery 170 and power assembly 172 for providing power to the device. In some embodiments, microfluidic device 100 contains board pads 174, mainboard 176, board retention unit 178, sensor board 180, and peel tab 182. In some embodiments, sensor board 180 is adjacent to sensor 120. In some embodiments, sensor board 180 is directly attached to sensor 120. It should be noted that the arrangement of the exemplary components described in conjunction with microfluidic device 100 are merely one possible configuration of the components, as other configurations are possible and therefore these examples are not intended to be limiting.

As described above, housings (e.g., a top housing, a bottom housing) may form an enclosure to enclose components described herein. For example, as schematically illustrated in FIG. 1C, top housing 130 and bottom housing 140 have been joined (relative to FIG. 1B) to enclose the components of microfluidic device 100 (e.g., the fluidic stack, the sensor); however, as schematically illustrated in FIG. 1C, at least a portion of the components of fluidic device 100 may remain exposed, such as a portion of the fluidic stack 110 (e.g., exposing the filter), in order to provide contact to a sample (not pictured).

The microfluidic components may provide one or more channels. These channels may serve a variety of functions, including to allow a fluid or serum to flow from one part of a device to another. Channels may be formed by and/or within layers or by at least a portion of the layer and/or surfaces, and channels (e.g., a first channel) may be in fluid communication with other channels (e.g., a second channel). In some embodiments, a first channel passes through the fluidic block from the top surface and to the bottom surface. For example, in some embodiments, a layer comprising at least a portion of a second channel may have the second channel in fluid communication with the first channel. In embodiments, the second channel is at least partially inscribed in the layer. In some embodiments, a portion of the bottom surface of the fluidic block defines at least a portion of the second channel. For certain embodiments, a top surface of the sensor element defines at least a portion of the second channel. In some cases, horizontal boundaries of the second channel are at least partially defined by a cut-out of the layer.

The fluidic components may include various surfaces and/or features used to control the flow of a sample. The location and position of these surfaces may provide one or more dimensions of a channel, may provide access to particular channel and/or may position a component (e.g., a sensor) in relation to another component (e.g., a fluid reservoir). The fluidic components may provide various layers of the fluidic stack. These layers may comprise any suitable material to achieve the techniques described herein, and may also be positioned to form channels or to provide contact with surfaces or other components of devices or methods described herein.

In some embodiments, a layer and/or a surface may have a particular thickness. In some embodiments, the thickness of a layer and/or a surface is no greater than 100 microns, no greater than 50 microns, no greater than 40 microns, no greater than 30 microns, no greater than 20 microns, no greater than 10 microns, no greater than 5 microns, or no greater than 1 micron. In some embodiments, the thickness of a layer and/or a surface is at least 1 micron, at least 5 microns, at least 10 microns, at least 20 microns, at least 30 microns, at least 40 microns, at least 50 microns, or at least 100 microns. Combinations of the above-reference range are possible (e.g., no greater than 50 μm and at least 5 μm). Other ranges are possible.

In some embodiments, adhesive layers (e.g., a first adhesive layer, a second adhesive layer and/or a third adhesive layer) may be present, but may be optional. These layers may join other components (e.g., a fluidic block with a filter). In some embodiments, a first adhesive layer is disposed above the filter. In some embodiments, a second adhesive layer disposed below the filter. In some embodiments, a third adhesive layer is disposed below the microfluidic block. In some embodiments, one or more of the adhesive layers are substantially rectangular.

In some embodiments, an adhesive layer (e.g., a first adhesive layer, a second adhesive layer) may have a particular thickness. In some embodiments, the thickness of an adhesive layer no greater than 100 microns, no greater than 50 microns, no greater than 40 microns, no greater than 30 microns, no greater than 20 microns, no greater than 10 microns, no greater than 5 microns, or no greater than 1 micron. In some embodiments, the thickness of an adhesive layer (e.g., a first adhesive layer, a second adhesive layer) is at least 1 micron, at least 5 microns, at least 10 microns, at least 20 microns, at least 30 microns, at least 40 microns, at least 50 microns, or at least 100 microns. Combinations of the above-reference range are possible (e.g., no greater than 50 μm and at least 5 μm, no greater than 70 μm and at least 3 μm, and/or the like). Other ranges are possible.

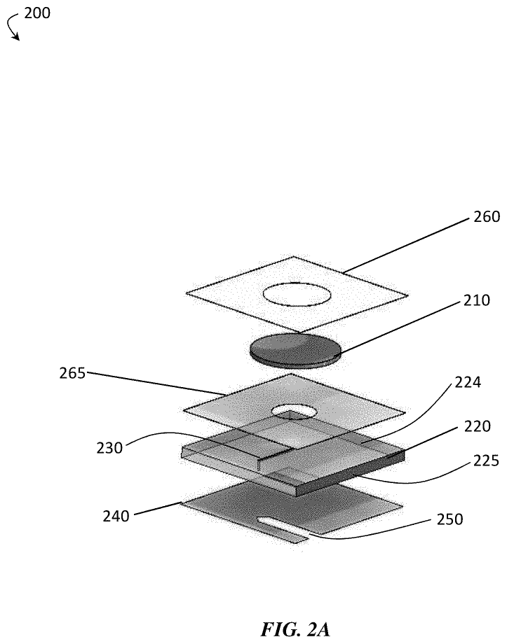

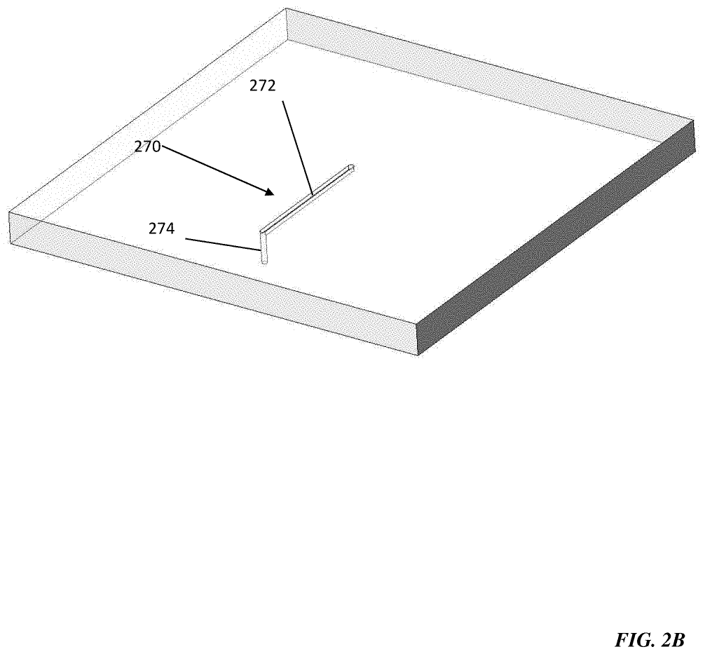

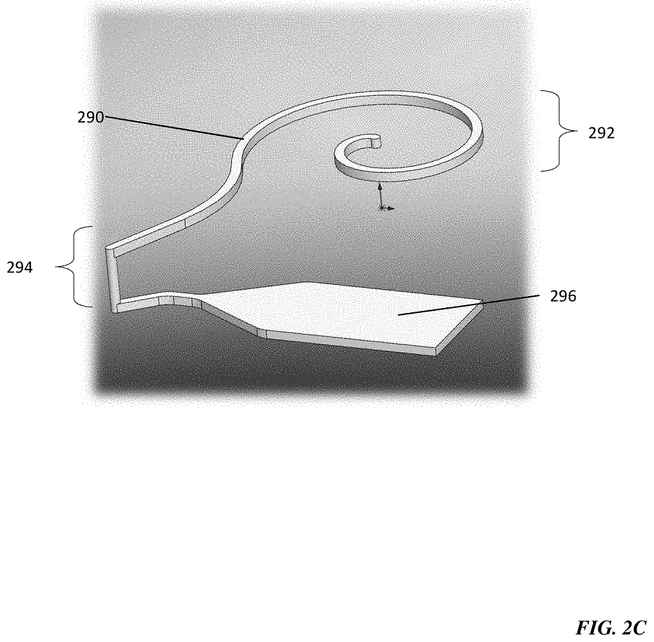

Referring now to FIG. 2A, an exemplary fluidic stack 200 is schematically illustrated, comprising filter 210 adjacent to fluidic block 220. In certain embodiments, filter 210 may be configured to remove red blood cells (i.e., a red blood cell filter) from a sample to produce a serum. Fluidic block 220, adjacent to filter 210, comprises a top surface 224 and a bottom surface 225 through which first channel 230 passes between. The serum may pass from filter 210 through first channel 230. First channel 230 is one example of an intervening channel, as it passes from a top surface (e.g., top surface 224) to a bottom surface (e.g., bottom surface 225) of the fluidic block. In some embodiments, first channel 230 is in fluid communication with filter 210 so that a sample (e.g., a serum) may pass from filter 210 to layer 240. As shown in FIG. 2A, layer 240 includes an empty portion that provides the sides of second channel 250. As described further herein, other dimensions of the second channel 250 can be provided by other components of the device (e.g., the top of the second channel 250 can be provided by the bottom side 225 of the fluidic block 220, and the bottom of second channel 250 can be provided by the top side of a sensor device disposed below layer 240).

In some embodiments, second channel 250 is in fluid communication with first channel 230, such that a sample may pass through filter 210, through first channel 230, and pass through second channel 250. In certain embodiments, fluidic stack 200 contains optional adhesive layers, such as first adhesive layer 260 and second adhesive layer 265, for spacing components, holding components in place, and/or joining components together, such as the filter and the fluidic block. For example, as schematically illustrated in FIG. 2A, second adhesive layer 265 is positioned between filter 210 and fluidic stack 220, which may be used to adhere filter 210 to top surface 224 of fluidic stack 220.

The fluidic block may contain various configurations of the intervening channel. In some embodiments, the fluidic block comprises a top surface, a bottom surface, and an intervening channel passing through the fluidic block from the top surface and to the bottom surface. The fluidic block, in some embodiments, is adjacent to the filter. In some embodiments, the fluidic block (or channel contained within the fluidic block) is in fluid communication with the filter. In some embodiments, the fluidic block is in fluid communication with another layer of the fluid stack (e.g., layer 240). In some embodiments still, the channel of the fluidic block is in fluid communication with a second channel (e.g., provided at least in part by the other layer of the fluid stack) that includes the sensor.

FIG. 2B shows a first exemplary embodiment of an intervening channel 270 of a fluidic block, according to some embodiments. In this example, the intervening channel 270 has a top portion 272 that extends along the top surface of the fluid block. The top portion 272 can be open to the top surface substantially along the entire length that extends across the top surface of the fluid block. The top portion can be open in such a manner to collect a sample or portion of a sample, such as a serum, from other fluidic stack components, such as from the filter. The intervening channel 270 further includes a second portion 274 that extends through the body of the fluid block to the bottom side of the fluid block. In this example, the second portion provides a microfluidic path for the fluid to travel from the top portion 272 to the bottom side of the fluid block. The opening of the second portion 274 of the intervening channel 270 can be disposed such that it is in contact with a second fluid channel (e.g., disposed such that it is located above the angled portion of the cutout 250 in layer 240).

FIG. 2C shows a second exemplary embodiment of an intervening channel 290 of a fluidic block, according to some embodiments. The intervening channel 290 includes a spiral portion 292 disposed at the top surface of the fluidic block (e.g., which is in fluid communication with the circular filter and therefore sized to be within the dimensions of the filter when assembled in the fluidic stack), a second portion 294 that extends through the thickness of the fluidic block, and a third portion 296 that provides top and side surfaces of a second channel (e.g., within which the sensor can be disposed). In such examples, an additional adhesive layer, such as layer 240, may not be included.

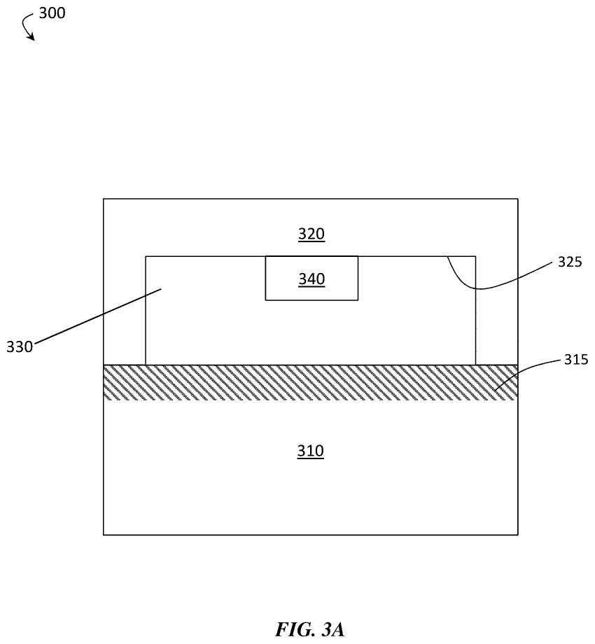

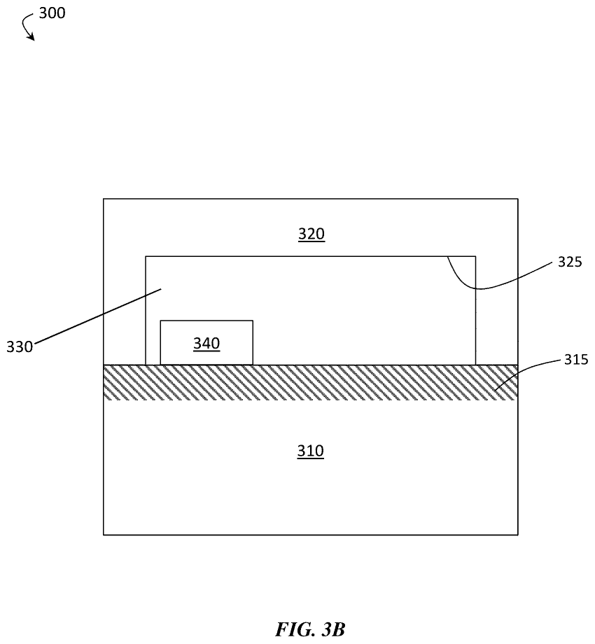

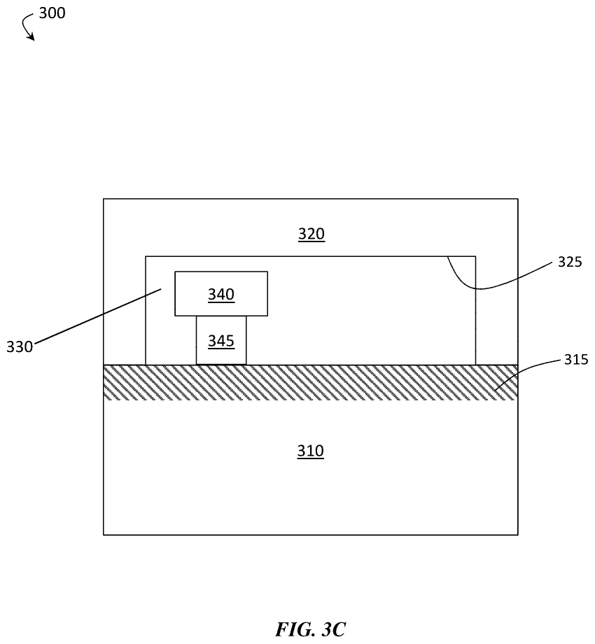

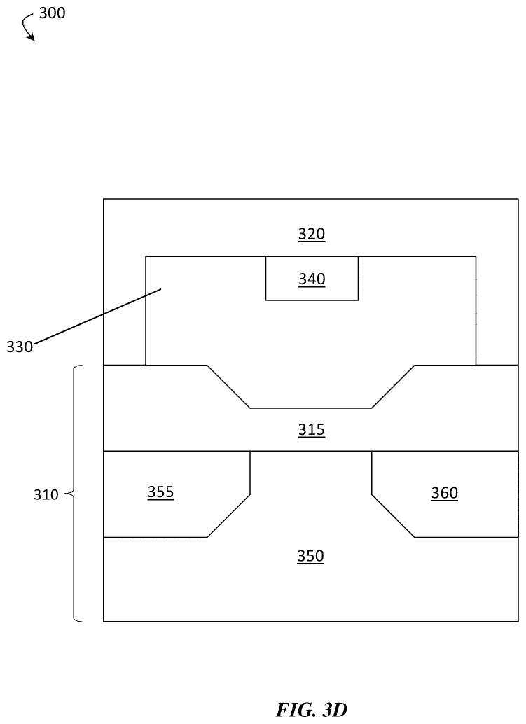

According to some embodiments, the biosensor device comprises a sensor. Referring now to FIG. 3A, device 300 comprises a sensor generally shown at 310 with a top surface 315. Device 300 also includes layer 320 with a bottom surface 325 (e.g., the fluidic block). As schematically illustrated in FIG. 3A, top surface 315 of sensor 310 and bottom surface 325 of layer 320 provide the top and bottom of the fluidic reservoir 330. While the sides of the fluidic reservoir 330 are shown as part of layer 320, this is for exemplary purposes, as a separate layer (e.g., layer 240) may be used to provide the sides of the fluidic reservoir 330.

A reference electrode, such as reference electrode 340, may be contained within fluidic reservoir 330. As described further herein, the reference electrode can be used when analyzing a sample for the presence of an analyte by serving as a reference measurement of the sample. In accordance with some embodiments, a reference electrode may have a variety of positions within devices as described herein. In some embodiments, a reference electrode is disposed within a fluidic reservoir. In some embodiments, the reference electrode is adjacent to the bottom surface of a layer of the fluidic stack. In some embodiments, the reference electrode is adjacent to the top surface of the sensor. The reference electrode may be connected to an anchor (e.g., a reference electrode anchor); that is to say, in some embodiments, an anchor mounts the reference electrode to a side of the chamber. In some cases, the anchor is adjacent to the top surface of the sensor. In some embodiments still, the second channel comprises at least a portion of the reference electrode and at least a portion of the sensor. Other positions of the reference electrode are possible.

FIGS. 3A-3D illustrate exemplary configurations of reference electrodes in a sensor device. FIG. 3A shows a first exemplary configuration with reference electrode 340 adjacent (e.g., directly adjacent) to bottom surface 325. FIG. 3B shows a second exemplary configuration with reference electrode 340 directly adjacent to top surface 315 of sensor 310. And FIG. 3C shows a third exemplary configuration with reference electrode 340 is adjacent to top surface 315, and is joined to top surface 315 by electrode anchor 345. As schematically illustrated in FIG. 3C, electrode anchor 345 is directly adjacent to top surface 315, but other configurations are possible, and electrode anchor 345 is optional and may not be present in certain embodiments.

Referring now to FIG. 3D, sensor 310 may comprise semiconductor 350, source 355, and drain 360, in addition to top surface 315. The arrangement and components of sensor 310 pictured in FIG. 3D may form a MOSFET. In certain embodiments, fluidic chamber 330 may contain an electrolyte or a serum comprising an analyte, which may bind to detectors disposed on the sensor and change the electrical properties (which can be measured using the source 355 and drain 360). It should be noted that the arrangement schematically illustrated in FIG. 3D is but one exemplary arrangement of a sensor, and other sensor arrangements or configurations are possible, as this disclosure is not so limited. In some embodiments, a back gate (not shown) is adjacent to the semiconductor.

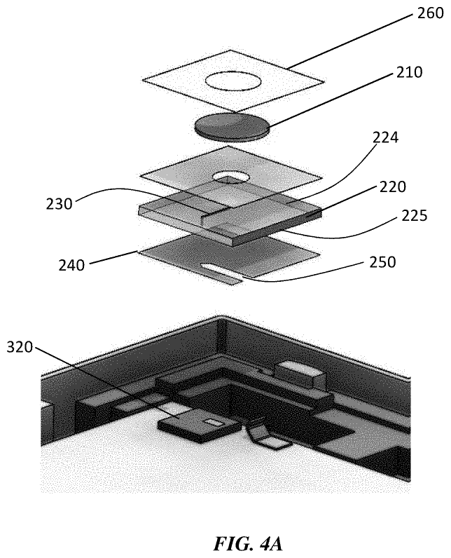

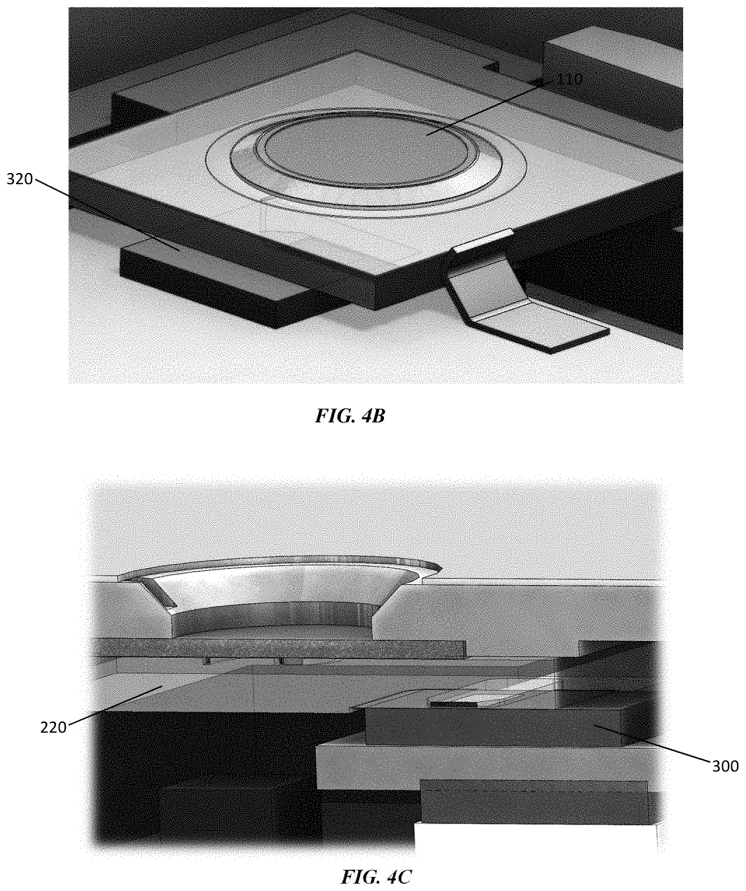

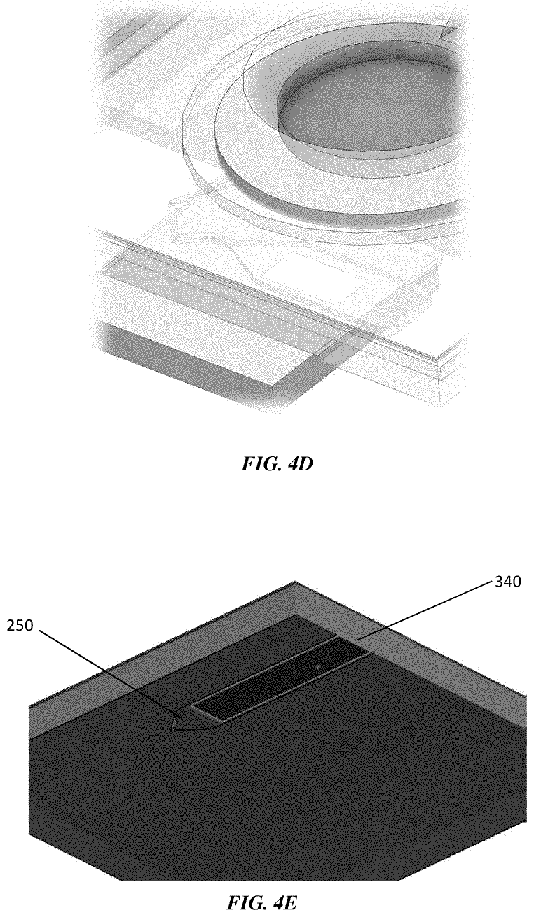

In some embodiments, one or more of the components of the fluidic stack may be adjacent to the sensor. Schematically illustrated in FIG. 4A, a portion of the fluidic stack 200 is adjacent to sensor 320. Fluidic stack 200 may be adjacent to layer 320 while leaving at least a portion of the fluidic stack, such as filter 310, exposed, as schematically illustrated in FIG. 4B. In some embodiments, fluidic block 220 is adjacent to at least a portion of device 300, shown in FIGS. 4C and 4D. In certain embodiments, when fluidic block 220 is directly adjacent to at least a portion of device 300, fluidic block 220 is in fluid communication with device 300 so that a sample (e.g., a serum) may pass from the fluidic stack 200, comprising fluidic block 220, for analysis by device 300. As shown in FIG. 4D, when the fluidic stack is in fluid communication (e.g., via second channel 250 of fluidic stack 200), reference electrode 340 may come in contact with a sample or analyte (not picture).

In use when analyzing a sample, a sample may pass through a filter to produce a serum, according to some embodiments. The filter may comprise any suitable material for filtering. A non-limiting example of a suitable material for filtering includes a polysulfone. That is to say that the filter may comprise polysulfone, a polysulfone membrane, and/or the like. The filter may also comprise any suitable shape for filtering. As a non-limiting example, the filter is circular (or substantially circular). According to some embodiments, the filter may remove red blood cells from the sample, such that a sample passing through the filter is free (or substantially free) of red blood cells. In some cases, a sample free (or substantially free) of red blood cells is the serum analyzed by the sensor. This filter may be in any suitable position for removing red blood cells from a sample prior to analysis by the sensor, but, in some embodiments, the filter is positioned adjacent to a fluidic block. In some embodiments, the filter is in fluid communication with the first channel. This may allow a sample passing through the filter (e.g., a serum) to proceed through the device towards the sensor.

In some embodiments, the serum is flowed from the filter through the intervening channel of the fluidic block. In some embodiments, the serum is flowed from the intervening channel through a second channel defined at least partially by another layer of the fluidic stack as described herein, wherein the layer is adjacent to the fluidic block and wherein the second channel is in fluid communication with the first channel. In some embodiments still, the serum is analyzed in the second channel using a sensor and a reference electrode, wherein the second channel comprises at least a portion of the reference electrode and at least a portion of the sensor.

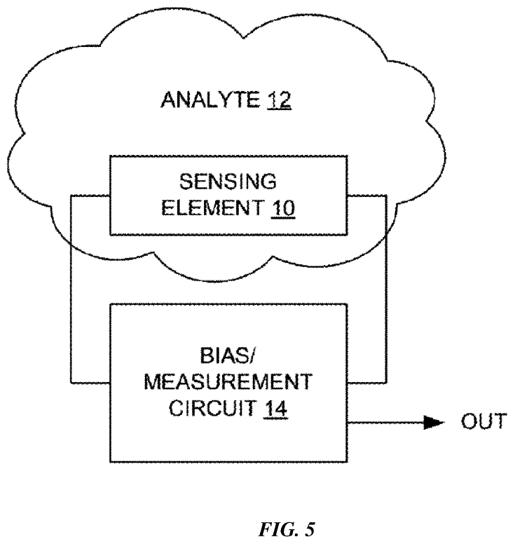

As described above and elsewhere herein, a device may comprise a sensor used to detect or measure a property of the sample. In some embodiments, the sensor comprises a top surface. This top surface may comprise an oxide or an oxide layer. In some embodiments, the sensor is a nanochannel sensor. In some embodiments, the sensor is a nanochannel-based sensor. In yet another embodiment, the sensor comprises a sensing-element. An non-limiting example of a nanochannel-based sensor with a sensing element is described below in Example 1 with reference to FIGS. 5-7.

The sensor may be positioned in a variety of positions. In some embodiments, a sensor is adjacent to the layer that provides a portion of the second channel (e.g., layer 240). In some embodiments, the sensor is adjacent to the layer and in fluid communication with the second channel. In some embodiments, the sensor may be positioned such that a top surface of the sensor defines at least a portion of the second channel (e.g., the bottom). That is to say, a sensor may comprise a top surface and the top surface of the senor may define at least a portion of the second channel, according to some embodiments. In some embodiments still, the sensor is adjacent to a back gate. Other positions of the sensor are possible. The sample (e.g., the serum) can be analyzed in the second channel using the sensor.

As described herein, the sensor can include a metal-oxide semiconductor field effect transistor. As understood by those skilled in the art, a MOSFET is a type of insulated-gate field-effect transistor that may be fabricated by the oxidation of a semiconductor whereby the voltage of the covered gate determines the electrical conductivity of the device, which may be used to change the conductivity with the amount of applied voltage and can be used for amplifying or switching electronic signals. Thus, according to some embodiments, the sensor may detect an analyte by a change in conductance through the semiconductor. In some embodiments, the sensor comprises a semiconductor, a source, and a drain. In some embodiments, the sensor further comprises a back gate. In some embodiments, the sensor comprise a plurality of antibodies to functionalize the sensor, as described further in conjunction with Example 1.

Devices and methods describe herein may comprise a reference electrode. As understood by those skilled in the art, a reference electrode provides a stable redox couple (i.e., oxidation-reduction reaction of a known electrochemical potential) by which other voltages may be referenced. In some embodiments, a voltage (or a related current) from a sensor (e.g., a semiconductor, a MOSFET) may be use or be in electronic communication with a reference electrode so that a voltage and/or an electronic signal (e.g., a conductance) can be determined. In some embodiments, the reference electrode comprises a silver/silver chloride (i.e., Ag/AgCl) reference electrode. The Ag/AgCl reference electrode can be governed by the following equations:

Ag⁺+e^{-←→Ag (}s)

AgCl (s)+e⁻←→Ag(s)+Cl⁻,

wherein the cell potential, E⁰, is 0.230 V±10 mV vs the standard hydrogen electrode.
However, other reference electrodes or redox couples are possible, as this disclosure is not so limited. An example of another reference electrode includes an iridium/iridium oxide reference electrode (e.g., Ir/IrO₄).

In some embodiments, a hermetic seal is provided. The hermetic seal may prevent leakage of a liquid or fluid, for example, from the fluid reservoir. In some embodiments, a hermetic seal is adjacent to a portion of the top surface, a portion of the bottom surface, and a portion of the fluid reservoir.

The following examples are intended to illustrate certain embodiments of the present techniques, but do not exemplify the full scope and should therefore not be considered limiting.

### EXAMPLE 1

As described herein, small-scale sensors, such as nanochannel-based sensors, can be used to detect an analyte in a liquid. FIG. 5 is a schematic diagram illustrating the use of a sensor device used to detect species in an analyte solution, according to some examples. In FIG. 5, a sensing element 10 is exposed to chemical or biological species(analyte) in an analyte solution 12. The sensing element 10 has connections to a bias/measurement circuit 14 that provides a bias voltage to the sensing element 10 and measures the differential conductance of the sensing element 10 (e.g., the small-signal change of conductance with respect to bias voltage). The differential conductance of the device is measured by applying a small modulation of bias voltage to generate a value of an output signal (OUT) that provides information about the chemical or biological species of interest in the analyte solution 12, for example a simple presence/absence indication or a multi-valued indication representing a concentration of the species in the analyte solution 12.

Suitable sensing elements (e.g., including semiconductor nanowires) and sensing technologies have been described in commonly-owned International Publication Number WO 2016/089,453 and U.S. Pat. No. 10,378,044, both of which are incorporated herein by reference in their entireties.

The sensing element 12 includes one or more elongated conductors of a semiconductor material such as silicon, which may be doped with impurities to achieve desired electrical characteristics. The dimensions of a channel can be sufficiently small (e.g., nanoscale) such that chemical/electrical activity on the channel surface can have a much more pronounced effect on electrical operation than in larger devices. Such nanoscale channels may be referred to as nanochannels herein. In some embodiments, the sensing element 12 has one or more constituent nanochannels having a cross-sectional dimension of less than about 150 nm (nanometers), and even more preferably less than about 100 nm.

As described herein, the surface of the sensing element 12 can be functionalized by using a series of chemical reactions to incorporate receptors or sites for chemical interaction with the species of interest in the analyte solution 12. As a result of this interaction, the charge distribution, or surface potential, of the surface of the sensing element 12 changes in a corresponding manner. Such a change of surface potential can alter the conductivity of the sensing element 10 in a way that is detected and measured by the bias/measurement circuit 14. Thus, the sensing element 12 can operate as a field-effect device, since the channel conductivity can be affected by a localized electric field related to the surface potential or surface charge density. The measured differential conductance values can be converted into values representing the property of interest (e.g., the presence or concentration of species), based on known relationships as may have been established in a separate calibration procedure, for example.

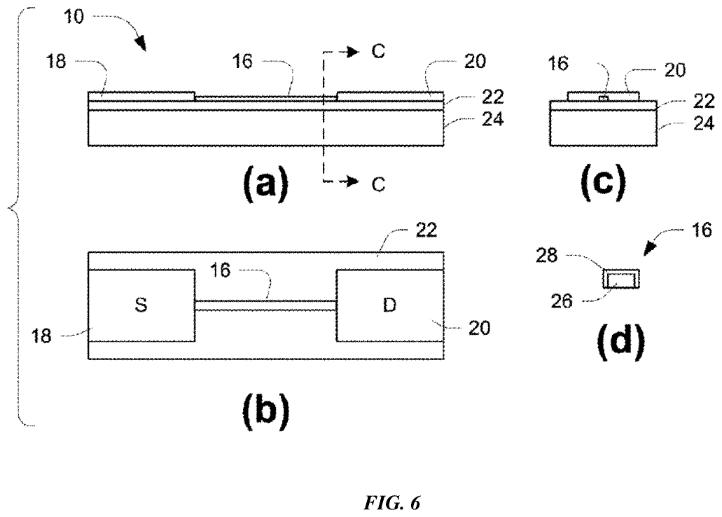

FIG. 6 shows a sensing element 10 according to one example. As shown in the side view of FIG. 6(a), a silicon nanochannel 16 extends between a source (S) contact 18 and a drain (D) contact 20, all formed on an insulating oxide layer 22 above a silicon substrate 24. FIG. 6(b) is a top view showing the narrow elongated nanochannel 16 extending between the wider source and drain contacts 18, 20, which are formed of a conductive material such as gold-plated titanium for example. FIG. 6(c) shows the cross-sectional view in the plane C-C of FIG. 6(a). FIG. 6(d) shows the cross section of the nanochannel 16 in more detail. In the illustrated embodiment, the nanochannel 16 includes an inner silicon member 26 and an outer oxide layer 28 such as aluminum oxide.

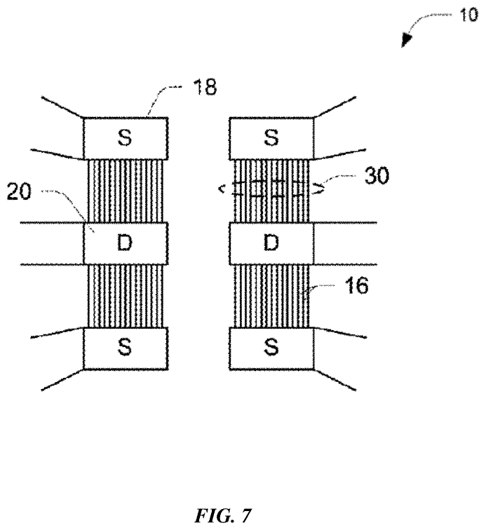

FIG. 7 shows a sensing element 10 employing an array of nanochannels 16, which in the illustrated example are arranged into four sets 30, each set including approximately twenty parallel nanochannels 16 extending between respective source and drain contacts 18, 20. By utilizing arrays of nanochannels 16 such as shown, greater signal strength (current) can be obtained, which can improve the signal-to-noise ratio of the sensing element 10. To obtain fully parallel operation, the source contacts 18 are all connected together by separate electrical conductors, and likewise the drain contacts 20 are connected together by separate electrical conductors. Other configurations are of course possible. For example, each set 30 may be functionalized differently so as to react to different species which may be present in the analyte solution 12, enabling an assay-like operation. In such configurations, it should be understood that each set 30 has separate connections to the bias/measurement circuit 14 to provide for independent operation.

The sensing element 10 may be made by a variety of techniques employing generally known semiconductor manufacturing equipment and methods. In some embodiments, Silicon-on-Insulator (SOI) wafers are employed. A starting SOI wafer may have a device layer thickness of 100 nm and oxide layer thickness of 380 nm, on a 600 μm boron-doped substrate, with a device-layer volume resistivity of 10-20 Ω-cm. After patterning the nanochannel channels and the electrodes (e.g., in separate steps), the structure can be etched out with an anisotropic reactive-ion etch (RIE). This process can expose the three surfaces (top and sides) of the silicon nanochannels 16 along the longitudinal direction, resulting in increased surface-to-volume ratio. A layer of Al2O3 (e.g., approximately 5 to 15 nm thick) can be grown using atomic layer deposition (ALD). Selective response to specific biological or chemical species can be realized by fabricating the nanochannels 16 such that once functionalized, the nanochannels 16 react to one or more analytes. In use, a flow cell, such as a machined plastic flow cell, can be employed. For example, a machined plastic flow cell can be fitted to the device and sealed with silicone gel, with the sensing element 10 bathed in a fluid volume (of about 30 μL for example), connected to a syringe pump.

In some embodiments, the sensing element 10 may include other control elements or gates adjacent to the nanochannels 16. For example, the sensing element 10 can include a top gate, which can be a conductive element formed along the top of each nanochannel 16. Such a top gate may be useful for testing, characterization, and/or in some applications during use, to provide a way to tune the conductance of the sensing element in a desired manner. As another example, the sensing element 10 may include one or more side gates formed alongside each nanochannel 16 immediately adjacent to the oxide layer 28, which can be used for similar purposes as a top gate. As a further example, in some embodiments the sensing element 10 can include a temperature sensor (e.g., disposed near the nanochannels). The system can use measurements from the temperature sensor to modify the system operations. For example, the circuitry can be configured to adjust how the system maps measured nanowire conductances to the concentration of an analyte. Further details on nanochannel sensors can be found in, for example, U.S. Patent Publication No. 2014/0030747, entitled “Nanochannel-based sensor system for use in detecting chemical or biological species,” which is attached hereto as Appendix A and incorporated by reference herein in its entirety.

### EXAMPLE 2

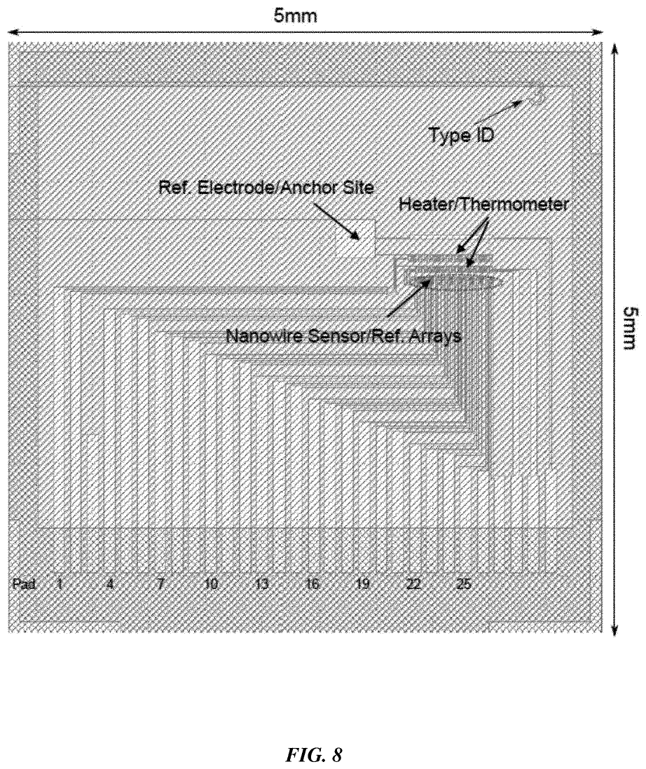

This example shows how a reference electrode/anchor site is configured into a main board comprising a nanowire sensor/ref. array, as shown in FIG. 8.

While several embodiments have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the techniques described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present techniques is/are used. Those skilled in the art will recognize, or be able to ascertain using no more than routine experimentation, many equivalents to the specific embodiments of the techniques described herein. It is, therefore, to be understood that the foregoing embodiments are presented by way of example only and that, within the scope of the appended claims and equivalents thereto, the techniques may be practiced otherwise than as specifically described and claimed. The techniques are directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present techniques. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A device, comprising:
a filter;
a fluidic block adjacent to the filter, the fluidic block comprising a top surface, a bottom surface, and a first channel passing through the fluidic block from the top surface and to the bottom surface;
a layer adjacent to the fluidic block, the layer comprising at least a portion of a second channel, wherein the second channel is in fluid communication with the first channel; and
a sensor adjacent to the layer and in fluid communication with the second channel.

2. The device of claim 1, wherein the filter is in fluid communication with the first channel.

3. The device of claim 1, wherein the second channel is at least partially inscribed in the layer.

4. The device of claim 1, wherein a portion of the bottom surface of the fluidic block defines at least a portion of the second channel.

5. The device of claim 1, wherein a top surface of the sensor defines at least a portion of the second channel.

6. The device of claim 1, wherein the sensor comprises a semiconductor.

7. The device of claim 1, wherein the sensor comprises a metal oxide semiconductor field effect transistor.

8. The device of claim 1, wherein the filter is a red blood cell filter.

9. The device of claim 1, wherein the filter is substantially circular.

10. The device of claim 1, further comprising a first adhesive layer disposed above the filter.

11. The device of claim 1, further comprising a second adhesive layer disposed below the filter.

12. The device of claim 1, wherein one or more of the layer, the first adhesive layer, and the second adhesive layer are substantially rectangular.

13. The device of claim 1, wherein a first dimension of the first adhesive layer, a second dimension of the second adhesive layer, or both, are sized to be larger than a third dimension of the filter.

14. The device of claim 1, wherein horizontal boundaries of the second channel are at least partially defined by a cut-out of the layer.

15. The device of claim 1, wherein the sensor comprises the device of claim 16.

16. A device, comprising:
a sensor comprising a top surface;
a layer comprising a bottom surface;
a fluid reservoir proximate the bottom surface of the layer and the top surface of the sensor; and
a reference electrode disposed within the fluid reservoir.

17-29. (canceled)

30. A device, comprising:
a fluidic stack, the fluidic stack comprising,
a filter;
a fluidic block adjacent to the filter, the fluidic block comprising a first surface, a second surface, and a first channel through the fluidic block from the first surface and to the second surface;
a first layer adjacent to the fluidic block, the first layer comprising at least a portion of a second channel, wherein the second channel is in fluid communication with the first channel; and
a sensor adjacent to the first layer and in fluid communication with the second channel; and
a fluidic chamber, the fluidic chamber comprising:
the sensor, the sensor further comprising a third surface; a second layer comprising a fourth surface;
a fluid reservoir proximate the third surface of the second layer and the fourth surface of the sensor; and
a reference electrode disposed within the fluid reservoir.

31. A method of analyzing a sample, comprising:
passing the sample through a filter to produce a serum;
flowing the serum through a fluidic block adjacent to the filter, the fluidic block comprising a top surface, a bottom surface, and a first channel through which the serum flows;
flowing the serum through a second channel of a layer, wherein the layer is adjacent to the fluidic block and wherein the second channel is in fluid communication with the first channel; and
analyzing the serum in the second channel using a sensor and a reference electrode, wherein the second channel comprises at least a portion of the reference electrode and at least a portion of the sensor.
